(19) United States
(12) Patent Application Publication
(10) Pub. No.: US 2003/0158094 A1
(43) Pub. Date: Aug. 21, 2003

(54) Combination product of an aryl-substituted propanolamine derivative with at least one other active ingredient and the use of the product

(76) Inventors: Heiner Glombik, Hofheim (DE); Wendelin Frick, Hunstetten-Beuerbach (DE); Hans-Ludwig Schafer, Hochheim (DE); Werner Kramer, Mainz-Laubenheim (DE)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/225,802

(22) Filed: Aug. 22, 2002

(30) Foreign Application Priority Data
Aug. 22, 2001 (DE) 10140170.1
Aug. 31, 2001 (DE) 10142455.8

(51) Int. Cl. A61K 38/14; A61K 31/7052; C07K 9/00

(52) U.S. Cl. 514/8; 514/23; 530/322; 536/17.4

(57) ABSTRACT

The present invention is directed to an aryl-substituted propanolamine derivative of formula I

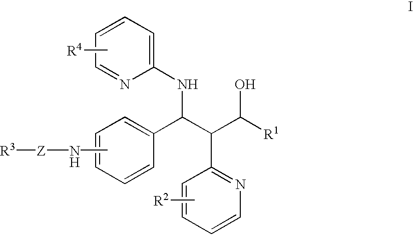

as described herein, a pharmaceutically acceptable salt or physiologically functional derivatives thereof, with an other active ingredient, or a pharmaceutically acceptable salt or physiologically functional derivatives thereof. The invention is also directed to the use of the combination product, pharmaceutical composition comprising the combination product and method for preparing the pharmaceutical composition.

### FIELD OF THE INVENTION

The present invention relates to a combination product of an aryl-substituted propanolamine derivative of formula I as described herein, with at least one other active ingredient

### BACKGROUND OF THE INVENTION

Aryl-substituted propanolamine derivatives of formula I having hypolipidemic effects have been described in EP 1 117 645.

Antidiabetics are described in the Rote Liste 2001, chapter 12. More specifically, the antidiabetics include insulin and insulin derivatives such as, for example, Lantus® (see www.lantus.com) or fast-acting insulins (see U.S. Pat. No. 6,221,633), GLP-1 derivatives such as, for example, those disclosed in WO 98/08871, and orally active hypoglycemic active ingredients. The orally active hypoglycemic active ingredients include, preferably, sulfonylureas, biguanides, meglitinides, oxadiazolidinediones, thiazolidinediones, glucosidase inhibitors, glucagon antagonists, GLP-1-agonists, potassium channel openers such as, for example, those disclosed in WO 97/26265 and WO 99/03861, insulin sensitizers, inhibitors of liver enzymes involved in the stimulation of gluconeogenesis and/or glycogenolysis, modulators of glucose uptake, compounds which alter lipid metabolism, such as antihyperlipidemic active ingredients and antilipidemic active ingredients, compounds which reduce food intake, PPAR and RXR agonists and active ingredients which act on the ATP-dependent potassium channel of the beta cells.

### SUMMARY OF THE INVENTION

The present invention is directed to an aryl-substituted propanolamine derivative of formula I

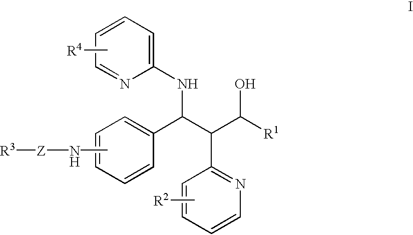

in which

R¹ is phenyl optionally substituted by one to three mutually independent radicals, or heteroaryl optionally substituted by one to three mutually independent radicals, where the radicals are selected from the group consisting of fluorine, chlorine, bromine, iodine, OH, CF₃, —NO₂, —CN, (C₁-C₈)-alkoxy, (C₁-C₈)-alkyl, —NH₂, —NH—R⁹, —N(R⁹)R¹⁰, —CHO, —COOH, —COOR¹¹, —(C═O)—R¹², (C₁-C₆)-alkyl-OH, (C₁-C₆)-alkyl(-OH)-phenyl, (C₁-C₆)-alkyl-CF₃, (C₁-C₆)-alkyl-NO₂, (C₁-C₆)-alkyl-CN, (C₁-C₆)-alkyl-NH₂, (C₁-C₆)-alkyl-NH—R⁹, (C₁-C₆)-alkyl-N(R⁹)R¹⁰, (C₁-C₆)-alkyl-CHO, (C₁-C₆)-alkyl-COOH, (C₁-C₆)-alkyl-COOR¹¹, (C₁-C₆)-alkyl-(C═O)—R¹², —O—(C₁-C₆)-alkyl-OH, —O—(C₁-C₆)-alkyl-CF₃, —O—(C₁-C₆)-alkyl-NO₂, —O—(C₁-C₆)-alkyl-CN, —O—(C₁-C₆)-alkyl-NH₂, —O—(C₁-C₆)-alkyl-NH-R⁹, —O—(C₁-C₆)-alkyl-N(R⁹)R¹⁰, —O—(C₁-C₆)-alkyl-CHO, —O—(C₁-C₆)-alkyl-COOH, —O—(C₁-C₆)-alkyl-COOR¹¹, —O—(C₁-C₆)-alkyl-(C═O)-R¹², —N—SO₃H, —SO₂—CH₃, —O—(C₁-C₆)-alkyl-O—(C₁-C₆)-alkyl-phenyl, (C₁-C₆)-alkylthio, or pyridyl, and wherein one or more hydrogen atom(s) in the alkyl radical is optionally replaced by fluorine, and wherein for the phenyl or pyridyl radical is optionally monosubstituted by methyl, methoxy or halogen;

R² is H, —OH, —CH₂OH, —OMe, —CHO, or —NH₂;

R³ is saccharide residue, disaccharide residue, trisaccharide residue, or tetrasaccharide residue, wherein the saccharide residue, disaccharide residue, trisaccharide residue or tetrasaccharide residue is optionally substituted by one or more saccharide protective groups selected from the group of HO—SO₂— and(HO)₂—PO—;

R⁴ is H, methyl, F, —OMe;

R⁹ to R¹² are, independently of one another, H or C₁-C₈-alkyl;

Z is —NH—C₀-C₁₆-alkyl-C═O—, —O—C₀-C₁₆-alkyl-C═O—, —(C═O)ₘ-C₁-C₁₆-alkyl-(C═O)ₙ—, amino acid residue, or diamino acid residue, wherein the amino acid residue or diamino acid residue is optionally substituted by one or more amino acid protective groups, or a covalent bond;

n is 0 or 1;or

m is 0 or 1;and

a pharmaceutically acceptable salt or physiologically functional derivatives thereof, with an other active ingredient, or a pharmaceutically acceptable salt or physiologically functional derivatives thereof. The invention is also directed to the use of the combination product, pharmaceutical composition comprising the combination product and method for preparing the pharmaceutical composition.

### DETAILED DESCRIPTION OF THE INVENTION

Definitions of Terms

As used above, and throughout the description of the invention, the following terms, unless otherwise indicated, shall be understood to have the following meanings.

“Pharmaceutically acceptable salt”, because of its greater solubility in water compared with the initial compound, means a salt that is particularly suitable for medical applications. The salt must have a pharmaceutically acceptable anion or cation. A suitable pharmaceutically acceptable salt as an acid addition salt of a compound of the invention, for example, is a salt of an inorganic acid such as hydrochloric acid, hydrobromic, phosphoric, metaphosphoric, nitric, sulfamic or sulfuric acid, or of organic acids such as, for example, acetic, benzenesulfonic, benzoic, citric, ethanesulfonic, fumaric, gluconic, glycolic, isethionic, lactic, lactobionic, maleic, malic, methanesulfonic, succinic, p-toluenesulfonic, tartaric or trifluoroacetic acid. The chloride salt is particularly preferably used for medical purposes. A suitable pharmaceutically acceptable salt as a base addition salt of a compound of the invention, for example, is an ammonium, alkali metal (such as sodium and potassium) and alkaline earth metal (such as magnesium and calcium) salt.

A salt with a pharmaceutically unacceptable anion likewise belongs in the scope of the invention as useful intermediates for the preparation or purification of a pharmaceutically acceptable salt or for use in nontherapeutic, for example in vitro, applications.

“Patient” means a mammal including a human.

“Physiologically functional derivative” means any physiologically tolerated derivative of a compound of the invention, e.g., a prodrug such a as an ester that is able on administration to a patient, to form (directly or indirectly) such a compound or an active metabolite thereof.

A prodrug of the compounds according to the invention are another aspect of this invention. Such prodrugs can be metabolized in vivo to give a compound according to the invention. These prodrugs may or may not be active themselves.

The a derivative of formula I can also exist in various polymorphous forms, for example as amorphous and crystalline polymorphous forms. All the polymorphous forms of the compounds according to the invention are included in the scope of the invention and are a further aspect of the invention.

All references to “compound(s) according to formula (I)” in the following text relate to compound(s) of formula I as described above and their salts, solvates and physiologically functional derivatives as described herein.

Heteroatoms particularly suitable in the abovementioned heteroaryl group are, for example, O, S, N.

Heteroaroyl groups, unless otherwise defined, have 1-15 carbon atoms and 1-6 heteroatoms; preferably 1-5 carbon atoms and 1-2 heteroatoms. Examples of suitable heteroaryl groups mentioned in the foregoing definitions are thiophene, furan, pyridine, pyrimidine, indole, quinoline, oxazole, isoxazole, thiazole or isothiazole.

“Alkyl” means straight-chain or branched hydrocarbon chains.

“Saccharide residue” means a compound derived from an aldose and ketose which has 3 to 7 carbon atoms and may belong to the D or L series; these include an amino saccharide, sugar alcohol or saccharic acid. Examples of saccharide residues are glucose, mannose, fructose, galactose, ribose, erythrose, glyceraldehyde, sedoheptulose, glucosamine, galactosamine, glucuronic acid, galacturonic acid, gluconic acid, galactonic acid, mannonic acid, glucamine, 3-amino-1,2-propanediol, glucaric acid and galactaric acid. The saccharide residue may also be substituted or protected.

“Di-, tri, or tetrasaccharide” means a saccharide composed respectively of two, three or four saccharide units. Di-, tri-, or tetrasaccharides are produced by acetal-like linkage with 2 or more sugars. The linkages may moreover occur in the α or β form. Examples of the polysaccharides are lactose, maltose and cellobiose.

“Protected saccharide” means a saccharide protected preferably on the hydrogen atom of an OH group of the saccharide. A suitable protective group for a hydroxyl group of a saccharide include the following: benzyl, acetyl, benzoyl, pivaloyl, trityl, tert-butyldimethylsilyl, benzylidene, cyclohexylidene and isopropylidene protective group.

“Amino acid” means, e.g., the stereoisomeric forms, i.e., D or L forms, of the following compounds:

| | | |
|---|---|---|
| alanine | glycine | proline |
| cysteine | histidine | glutamine |
| aspartic acid | isoleucine | arginine |
| glutamic acid | lysine | serine |
| phenylalanine | leucine | threonine |
| tryptophan | methionine | valine |
| tyrosine | asparagine | |

| | |
|---|---|
| 2-aminoadipic acid | 2-aminoisobutyric acid |
| 3-aminoadipic acid | 3-aminoisobutyric acid |
| beta-alanine | 2-aminopimelic acid |
| 2-aminobutyric acid | 2,4-diaminobutyric acid |
| 4-aminobutyric acid | desmosine |
| piperidic acid | 2,2-diaminopimelic acid |
| 6-aminocaproic acid | 2,3-diaminopropionic acid |
| 2-aminoheptanoic acid | N-ethylglycine |
| 2-(2-thienyl)-glycine | 3-(2-thienyl)-alanine |
| penicillamine | sarcosine |
| N-ethylasparagine | N-methylisoleucine |
| hydroxylysine | 6-N-methyllysine |
| allo-hydroxylysine | N-methylvaline |
| 3-hydroxyproline | norvaline |
| 4-hydroxyproline | norleucine |
| isodesmosine | ornithine |
| allo-isoleucine | |
| N-methylglycine. | |

Abbreviated names for the amino acids follow the generally customary names (cf. Schröder, Lübke, The Peptides, Vol. I, New York 1965, pages XXII-XXIII; Houben-Weyl, Methoden der Organischen Chemie [Methods of Organic Chemistry], Volume XV/1 and 2, Stuttgart 1974). The amino acid pGlu is pyroglutamyl, Nal is 3-(2-naphthyl)alanine, azagly-NH2 is a compound of the formula NH2—HN—CONH2 and D-Asp is the D form of aspartic acid. According to their chemical nature, peptides are amides and decompose into amino acids on hydrolysis.

Diamino acid, triamino acid, and tetraamino acid residue mean peptides composed of 2 to 4 of the abovementioned amino acids.

A suitable “amino acid protective group” (see, for example, T. W. Greene, “Protective Groups in Organic Synthesis”) for an amino acid are mainly: t-butyloxycarbonyl (BOC), 9-fluorenylmethoxycarbonyl (Fmoc), benzyloxycarbonyl (Z), 2-(3,5-dimethoxyphenyl)prop-2-yloxycarbonyl (Ddz), methyl, t-butyl, trityl, and s-t-butyl..

“Other active ingredient” that is suitable for the combination products means: all antidiabetics such as those mentioned in the Rote Liste 2001, chapter 12. Most of the other active ingredients listed below are disclosed in the USP Dictionary of USAN and International Drug Names, US Pharmacopeia, Rockville 2001. More specifically, the antidiabetics include insulin and insulin derivatives such as, for example, Lantus® (see www.lantus.com) or fast-acting insulins (see U.S. Pat. No. 6,221,633), GLP-1 derivatives such as, for example, those disclosed in WO 98/08871, and orally active hypoglycemic active ingredients. The orally active hypoglycemic active ingredients include, preferably, sulfonylureas, biguanides, meglitinides, oxadiazolidinediones, thiazolidinediones, glucosidase inhibitors, glucagon antagonists, GLP-1-agonists, potassium channel openers such as, for example, those disclosed in WO 97/26265 and WO 99/03861, insulin sensitizers, inhibitors of liver enzymes involved in the stimulation of gluconeogenesis and/or glycogenolysis, modulators of glucose uptake, compounds which alter lipid metabolism, such as antihyperlipidemic active ingredients and antilipidemic active ingredients, compounds which reduce food intake, PPAR and RXR agonists and active ingredients which act on the ATP-dependent potassium channel of the beta cells.

Embodiments

A preferred embodiment of the invention is where the aryl-substituted propanolamine derivative of formula I and other active ingredient of the combination product displays an activity that is synergistic. Further preferred is where the hypolipidemic activity of the aryl-substituted propanolamine derivative of formula In the combination product is increased synergistically to a disproportionately large extent by the other active ingredient.

Another preferred embodiment of the invention is where the combination product comprises the derivative of formula I wherein:

R¹ is phenyl, thiazolyl, oxazolyl, and isoxazolyl, and wherein for the phenyl thiazolyl, oxazolyl, and isoxazolyl are optionally substituted by one to two fluorine, chlorine, bromine, or (C₁-C₈)-alkyl;

R³ is a saccharide residue selected from the group consisting of

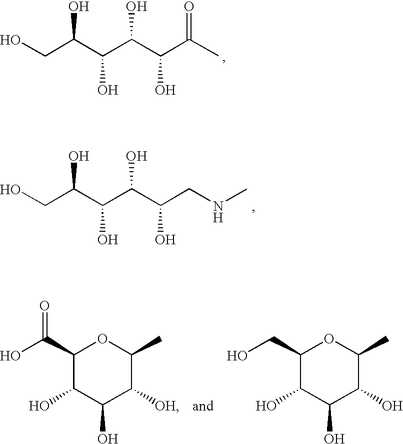

wherein the saccharide residue is optionally substituted by one or more HO—SO₂—; and

Z is —NH—C₆-C₁₂-alkyl-C═O—, —O—C₆-C₁₂-alkyl-C═O—, —(C═O)ₘ—C₆-C₁₂-alkyl-(C═O)ₙ—; or

the pharmaceutically acceptable acid addition salt thereof.

A further preferred embodiment of the invention is where the combination product comprises the derivative of the following formula

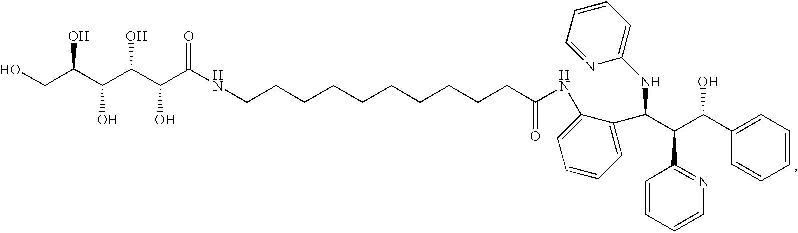

the pharmaceutically acceptable acid addition salt thereof.

Another preferred embodiment of the invention is where the other active ingredient is an orally active hypoglycemic.

Another particular embodiment of the invention is where the derivative of formula Is administered in combination with an HMG-CoA reductase inhibitor such as simvastatin, fluvastatin, pravastatin, lovastatin, atorvastatin, cerivastatin, or rosuvastatin.

Another particular embodiment of the invention is where the derivative of formula Is administered in combination with a cholesterol absorption inhibitor such as, for example, ezetimibe, tiqueside, or pamaqueside.

Another particular embodiment of the invention is where the derivative of formula Is administered in combination with a PPAR gamma agonist such as, for example, rosiglitazone, pioglitazone, JTT-501 (see Table II), or GI 262570 (see Table II).

Another particular embodiment of the invention is where the derivative of formula Is administered in combination with PPAR alpha agonists such as, for example, GW 9578 (see Table I), or GW 7647 (see Table I).

Another particular embodiment of the invention is where the derivative of formula Is administered in combination with a mixed PPAR alpha/gamma agonist such as, for example, GW 1536 (see Table I), AVE 8042, AVE 8134, or AVE 0847, or as described in PCT/US 11833, PCT/US 1 1490, or DE10142734.4.

Another particular embodiment of the invention is where the derivative of formula Is administered in combination with a fibrate such as, for example, fenofibrate, clofibrate, or bezafibrate.

Another particular embodiment of the invention is where the derivative of formula Is administered in combination with an MTP inhibitor such as, for example, implitapide, BMS 201038 (see Table I), or R 103757 (see Table I).

Another particular embodiment of the invention is where the derivative of formula Is administered in combination with bile acid absorption inhibitors (see, for example, U.S. Pat. No. 6,245,744 or U.S. Pat. No. 6,221,897) such as, for example, HMR 1741.

Another particular embodiment of the invention is where the derivative of formula Is administered in combination with a CETP inhibitor such as, for example, JTT-705 (see Table II).

Another particular embodiment of the invention is where the derivative of formula Is administered in combination with a polymeric bile acid adsorbent such as, for example, cholestyramine, or colesevelam.

Another particular embodiment of the invention is where the derivative of formula Is administered in combination with an LDL receptor inducer (see U.S. Pat. No. 6,342,512) such as, for example, HMR1171, or HMR1586.

Another particular embodiment of the invention is where the derivative of formula Is administered in combination with an ACAT inhibitor such as, for example, avasimibe.

Another particular embodiment of the invention is where the derivative of formula Is administered in combination with an antioxidant such as, for example, OPC 14117 (see Table II).

Another particular embodiment of the invention is where the derivative of formula Is administered in combination with a lipoprotein lipase inhibitor such as, for example, NO 1886 (see Table II).

Another particular embodiment of the invention is where the derivative of formula Is administered in combination with an ATP-citrate lyase inhibitor such as, for example, SB 204990 (see Table II).

Another particular embodiment of the invention is where the derivative of formula Is administered in combination with a squalene synthetase inhibitor such as, for example, BMS 188494 (see Table II).

Another particular embodiment of the invention is where the derivative of formula Is administered in combination with a lipoprotein(a) antagonist such as, for example, CI 1027 (see Table II) or nicotinic acid.

Another particular embodiment of the invention is where the derivative of formula Is administered in combination with a lipase inhibitor such as, for example, orlistat.

Another particular embodiment of the invention is where the derivative of formula Is administered in combination with insulin.

Another particular embodiment of the invention is where the derivative of formula Is administered in combination with a sulfonylurea such as, for example, tolbutamide, glibenclamide, glipizide or glimepiride.

Another particular embodiment of the invention is where the derivative of formula Is administered in combination with a biguanide such as, for example, metformin.

Another particular embodiment of the invention is where the derivative of formula Is administered in combination with a meglitinide such as, for example, repaglinide.

Another particular embodiment of the invention is where the derivative of formula Is administered in combination with a thiazolidinedione such as, for example, troglitazone, ciglitazone, pioglitazone, rosiglitazone or the compounds disclosed in WO 97/41097, in particular 5-[[4-[(3,4-dihydro-3-methyl-4-oxo-2-quinazolinylmethoxy]phenyl]methyl]-2,4-thiazolidinedione.

Another particular embodiment of the invention is where the derivative of formula Is administered in combination with an α-glucosidase inhibitor such as, for example, miglitol or acarbose.

Another particular embodiment of the invention is where the derivative of formula Is administered in combination with an active ingredient which acts on the ATP-dependent potassium channel of the beta cells, such as, for example, tolbutamide, glibenclamide, glipizide, glimepiride or repaglinide.

Another particular embodiment of the invention is where the derivative of formula Is administered in combination with more than one of the aforementioned compounds, e.g., in combination with a sulfonylurea and metformin, a sulfonylurea and acarbose, repaglinide and metformin, insulin and a sulfonylurea, insulin and metformin, insulin and troglitazone, insulin and lovastatin, etc.

Another further particular embodiment of the invention is where the derivative of formula Is administered in combination with CART modulators (see “Cocaine-amphetamine-regulated transcript influences energy metabolism, anxiety and gastric emptying in mice” Asakawa, A, et al., M.:Hormone and Metabolic Research (2001), 33(9), 554-558), NPY antagonists, e.g. naphthalene-1-sulfonic acid {4-[(4-aminoquinazolin-2-ylamino)methyl]cyclohexylmethyl}amide; hydrochloride (CGP 71683A)), MC4 agonists (e.g. 1-amino-1,2,3,4-tetrahydronaphthalene-2-carboxylic acids [2-(3a-benzyl-2-methyl-3-oxo-2,3,3a,4,6,7-hexahydropyrazolo[4,3-c]pyridin-5-yl)-1-(4-chlorophenyl)-2-oxo-ethyl]amide; (WO 01/91752)), orexin antagonists (e.g. 1-(2-methylbenzoxazol-6-yl)-3-[1,5]naphthyridin-4-ylurea; hydrochloride (SB-334867-A)), H3 agonists (3-cyclohexyl-1-(4,4-dimethyl-1,4,6,7-tetrahydroimidazo[4,5-c]pyridin-5-yl) propan-1-one oxalic acid salt (WO 00/63208)); TNF agonists, CRF antagonists (e.g. [2-methyl-9-(2,4,6-trimethylphenyl)-9H-1,3,9-triazafluoren-4-yl]dipropylamine (WO 00/66585)), CRF BP antagonists (e.g. urocortin), urocortin agonists, β3-agonists (e.g. 1-(4-chloro-3-methanesulfonylmethylphenyl)-2-[2-(2,3-dimethyl-1H-indol-6-yloxy)ethylamino]ethanol; hydrochloride (WO 01/83451)), MSH (melanocyte-stimulating hormone) agonists, CCK-A agonists (e.g. {2-[4-(4-chloro-2,5-dimethoxyphenyl)-5-(2-cyclohexylethyl)thiazol-2-ylcarbamoyl]-5,7- dimethylindol-1-yl}acetic acid trifluoroacetic acid salt (WO 99/15525)); serotonin reuptake inhibitors (e.g. dexfenfluramine), mixed serotoninergic and noradrenergic compounds (e.g. WO 00/71549), 5HT agonists e.g. 1-(3-ethylbenzofuran-7-yl)piperazine oxalic acid salt (WO 01/09111), bombesin agonists, galanin antagonists, growth hormone (e.g. human growth hormone), growth hormone-releasing compounds (6-benzyloxy-1-(2-diisopropylaminoethylcarbamoyl)-3,4-dihydro-1H-isoquinoline-2-carboxylic acid tert-butyl ester (WO 01/85695)), TRH agonists (see, for example, EP 0 462 884), uncoupling protein 2 or 3 modulators, leptin agonists (see, for example, Lee, Daniel W.; Leinung, Matthew C.; Rozhavskaya-Arena, Marina; Grasso, Patricia. Leptin agonists as a potential approach to the treatment of obesity. Drugs of the Future (2001), 26(9), 873-881), DA agonists (bromocriptine, Doprexin), lipase/amylase inhibitors (e.g. WO 00/40569), PPAR modulators (e.g. WO 00/78312), RXR modulators or TR-β agonists.

Another particular embodiment of the invention is where the other active ingredient is leptin, see, for example, “Perspectives in the therapeutic use of leptin”, Salvador, Javier; Gomez-Ambrosi, Javier; Fruhbeck, Gema, Expert Opinion on Pharmacotherapy (2001), 2(10), 1615-1622.

Another particular embodiment of the invention is where the other active ingredient is dexamphetamine or amphetamine.

Another particular embodiment of the invention is where the other active ingredient is fenfluramine or dexfenfluramine.

Another particular embodiment of the invention is where the other active ingredient is sibutramine.

Another particular embodiment of the invention is where the other active ingredient is orlistat.

Another particular embodiment of the invention is where the other active ingredient is mazindol or phentermine.

Another particular embodiment of the invention is where the derivative of formula Is administered in combination with dietary fiber materials, preferably insoluble dietary fiber materials (see, for example, Carob/Caromax® (Zunft H J; et al., Carob pulp preparation for treatment of hypercholesterolemia, ADVANCES IN THERAPY (2001 September-October), 18(5), 230-6.) Caromax is a carob-containing product supplied by Nutrinova, Nutrition Specialties & Food Ingredients GmbH, Industriepark Höchst, 65926 Frankfurt/Main)). Combination with Caromax® is possible in one preparation or by a separate administration of a derivative of formula I and Caromax®. Caromax® can moreover be administered in the form of foodstuffs such as, for example, in bakery products or muesli bars. Combination of a derivative of formula I with Caromax® not only improves the effect, in particular in LDL-cholesterol lowering, compared with the individual active ingredients, but is also tolerated better.

TABLE I

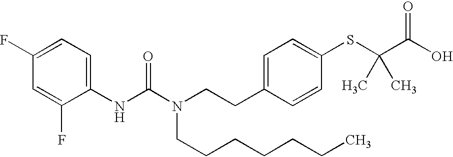

GW-9578

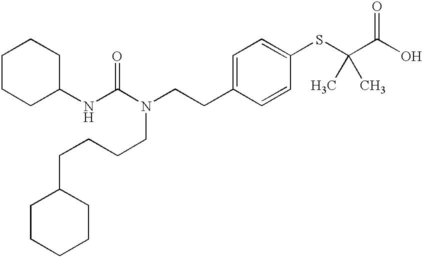

GW-7647

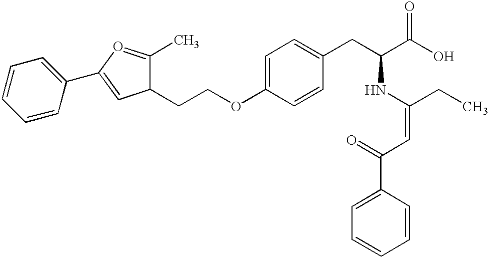

GW-1536

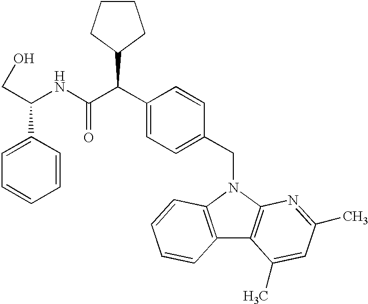

Implitapide

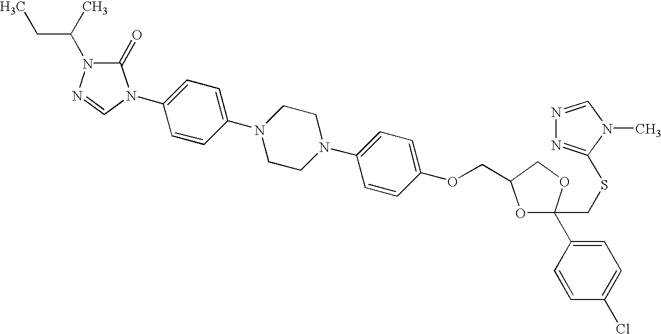

R-103757

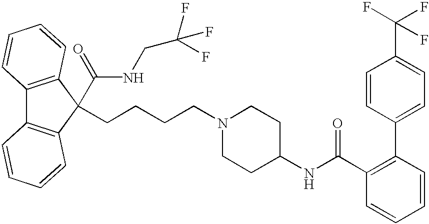

BMS-201038

TABLE II

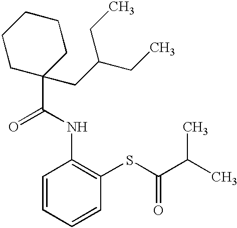

JTT-705

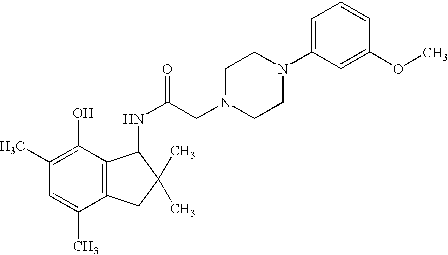

OPC-14177

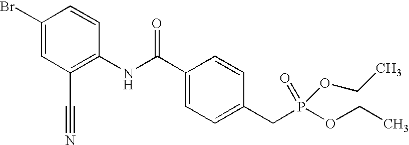

NO-1886

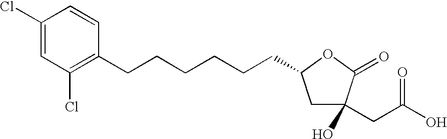

SB-204990

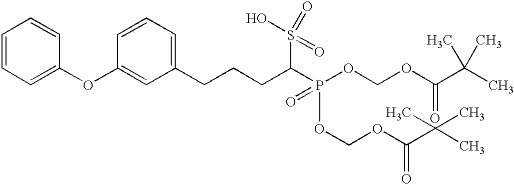

BMS-188494

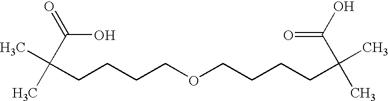

CI-1027

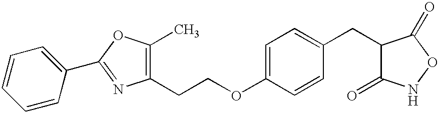

JTT-501

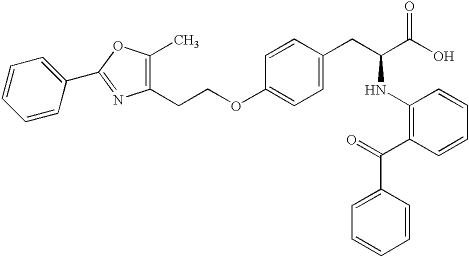

GI 262570

The amount of a derivative of formula I and of the other active ingredient necessary to achieve the desired biological effect with the combination product depends on a number of factors, e.g., the specific derivative of formula I or other active ingredient chosen, the intended use, the mode of administration and the clinical condition of the patient. The daily dose is generally in the range from 0.1 to 100 mg (typically from 0.1 to 50 mg) per day per kilogram of body weight, e.g. 0.1-10 mg/kg/day. Tablets or capsules may contain, for example, from 0.01 to 100 mg, typically from 0.02 to 50 mg. In the case of a pharmaceutically acceptable salt, the aforementioned weight data are based on the weight of the derivative of formula I or other active ingredient derived from the salt. The derivative of formula I or other active ingredient of the combination product, however, is preferably in the form of a pharmaceutical composition with a convertible carrier. The carrier must, of course, be compatible in the sense that it is compatible with the derivative of formula I or other active ingredient of the combination product and is not harmful for the patient's health. The carrier may be a solid or a liquid or both and is preferably formulated with the compound as single dose, for example as a tablet, which contain from 0.05% to 95% by weight of the other active ingredient. Other pharmaceutically active substances may likewise be present, including other derivative of formula I. The pharmaceutical combination product of the invention can be produced by one of the known pharmaceutical methods which consist essentially of mixing the derivative of formula I or other active ingredient with pharmacologically acceptable carriers and/or excipients.

The pharmaceutical combination product of the invention is one suitable for oral and peroral (e.g. sublingual) administration, although the most suitable mode of administration depends in each individual case on the nature and severity of the condition to be treated and on the nature of the particular derivative of formula I used. Coated formulations and coated slow-release formulations of the combination product are also within the scope of the invention. Acid- and gastric juice-resistant formulations are preferred. Suitable gastric juice-resistant coatings comprise cellulose acetate phthalate, polyvinyl acetate phthalate, hydroxypropylmethylcellulose phthalate and anionic polymers of methacrylic acid and methyl methacrylate.

Suitable pharmaceutical compositions comprising the derivative of formula I or other active ingredient for oral administration may be in the form of separate units such as, for example, capsules, cachets, lozenges or tablets, each of which contain a defined amount of the derivative of formula I and of the other active ingredient; as powders or granules; as a solution or suspension in an aqueous or nonaqueous liquid; or as an oil-in-water or water-in-oil emulsion. The combination product may, as already mentioned, be prepared by any suitable pharmaceutical method which includes a step in which the derivative of formula I or other active ingredient and the carrier (which may consist of one or more additional ingredients) are brought into contact. The combination product is generally produced by a uniform and homogeneous mixing of the derivative of formula I or other active ingredient with a liquid and/or finely divided solid carrier, after which the product is shaped if necessary. Thus, for example, a tablet can be produced by compressing or shaping a powder or granules of the compound, and the other ingredient. Compressed tablets may be produced by tableting the derivative of formula I or other active ingredient in free-flowing form, such as, for example, a powder or granules, where appropriate mixed with a binder, glidant, inert diluent and/or one (or more) surface-active/dispersing agent in a suitable machine. Shaped tablets can be produced by shaping the derivative of formula I or other active ingredient which is in powder form and has been moistened with an inert liquid diluent in a suitable machine.

Pharmaceutical composition products suitable for peroral (sublingual) administration include lozenges which contain a derivative of formula I and the other active ingredient with a flavoring, normally sucrose and gum arabic or tragacanth, and pastilles which comprise the derivative of formula I or other active ingredient in an inert base such as gelatin and glycerol or sucrose and gum arabic.

Other active ingredients may be combined with a derivative of formula In particular for synergistic improvement of the effect. Administration of the other active ingredient combination and the derivative of formula I can also take place either by separate administration of the other active ingredient and the derivative of formula I to the patient, i.e., an in vivo formation of a combination product, or in the form of a combination product in which the other active ingredient and derivative of formula I are present in one pharmaceutical preparation. When the administration of the other active ingredient combination and the compound of formula I takes place by separate administration such administration should be undertaken so that the effects of each combine additively or synergistically, preferably synergistically. Thus, the separate administration is preferably undertaken closely in time, e.g., within 10 minutes of each other.

The combined use of the derivative of formula I, and the other active ingredient are used for the therapeutic purposes noted herein in a patient, such that the combination is present in a pharmaceutically effective amount. That pharmaceutically effective amount arises from the use of the derivative of formula I and the other active ingredient wherein each is used in a pharmaceutically effective amount , or by virtue of additive or synergistic effects arising from the combined use, each can also be used in a subclinical pharmaceutically effective amount, i.e., an amount that, if used alone, provides for reduced or ineffective pharmaceutical effectiveness for the therapeutic purposes noted herein, provided that the combined use is pharmaceutically effective. In addition, the present invention encompasses the use of the combination of the derivative of formula I and the other active ingredient as described herein, where the derivative of formula I or the other active ingredient is present in a pharmaceutically effective amount, and the other is present in a subclinical pharmaceutically effective, provided that the combined use is pharmaceutically effective owing to their additive or synergistic effects. As used herein, the term “additive effect” describes the combined effect of two (or more) pharmaceutically active agents that is equal to the sum of the effect of each agent given alone. A syngergistic effect is one in which the combined effect of two (or more) pharmaceutically active agents is greater than the sum of the effect of each agent given alone. It is self-evident that every suitable combination of a derivative of formula I with one or more of the aforementioned other active ingredients and optionally with one or more other pharmacologically active substances is to be regarded as covered by the scope of protection of the present invention.

The combination products comprising a derivative of formula I represent ideal medicaments for the treatment of lipid metabolism disorders and/or carbohydrate metabolism disorders, especially hyperlipidemia or metabolic syndrome. The combination products are likewise suitable for modulating the decrease of the serum cholesterol level or for the prevention or treatment of arteriosclerotic manifestations.

The following preparations serve to illustrate the invention without, however, restricting it.

### EXAMPLE A

Soft gelatin capsules containing 100 mg of active ingredients per capsule:

| | per capsule |
|---|---|
| active ingredients | 100 mg |
| triglyceride mixture fractionated from coconut fat | 400 mg |
| capsule contents | 500 mg |

### EXAMPLE B

Emulsion containing 60 mg of active ingredients per 5 ml:

| | per 100 ml of emulsion |
|---|---|
| active ingredients | 1.2 g |
| neutral oil | q.s. |
| sodiumcarboxymethylcellulose | 0.6 g |
| polyoxyethylene stearate | q.s. |
| glycerol, pure | 0.2 to 2.0 g |
| flavoring | q.s. |
| water (deionized or distilled) | ad 100 ml |

### EXAMPLE C

Rectal drug form containing 40 mg of active ingredients per suppository:

| | per suppository |
|---|---|
| active ingredients | 40 mg |
| suppository base | ad 2 g |

### EXAMPLE D

Tablets containing 40 mg of active ingredients per tablet:

| | per tablet |
|---|---|
| lactose | 600 mg |
| corn starch | 300 mg |
| soluble starch | 20 mg |
| magnesium stearate | 40 mg |
| | 1000 mg |

### EXAMPLE E

Coated tablets containing 50 mg of active ingredients per coated tablet:

| | per coated tablet |
|---|---|
| active ingredients | 50 mg |
| corn starch | 100 mg |
| lactose | 60 mg |
| sec. calcium phosphate | 30 mg |
| soluble starch | 5 mg |
| magnesium stearate | 10 mg |
| colloidal silica | 5 mg |
| | 260 mg |

### EXAMPLE F

The following formulations are suitable for producing the contents of hard gelatin capsules:

| | | |
|---|---|---|
| a) | active ingredients | 100 mg |
| | corn starch | 300 mg |
| | | 400 mg |
| b) | active ingredients | 140 mg |
| | lactose | 180 mg |
| | corn starch | 180 mg |
| | | 500 mg |

### EXAMPLE G

Drops can be produced using the following formulation (100 mg of active ingredient in 1 ml=20 drops):

| | | |
|---|---|---|
| active ingredients | 10 | g |
| methyl benzoate | 0.07 | g |
| ethyl benzoate | 0.03 | g |
| ethanol, 96% | 5 | ml |
| demineralized water | ad 100 | ml |

Experimental

The synergistic activity of the combination product of a derivative of formula I with other active ingredients was tested in an animal experiment. For this purpose, the following compound C1 from the derivative of formula I was tested:

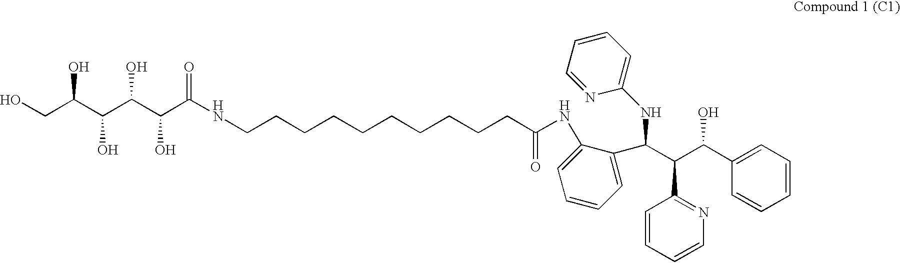

Hamsters were used for the biological testing of the combination product of the invention.

Male Syrian hamsters (Mesocricetus auratus) from 8 to 10 weeks of age were used for the experiment. The animals received a standard feed (Teklad 8604M) supplemented with 0.1% cholesterol. An additional normal control group received only standard feed.

The test substances were administered orally by gavage once a day on 12 consecutive days, and the control group was treated with the vehicle.

Feces were collected on days 5 and 6 of the experiment for bile acid analysis. Retroorbital blood was taken from the animals on day 10 of the experiment, and the lipid levels in the plasma were determined. Radioactive tracers were administered orally to the animals on day 11 of the experiment to determine the cholesterol absorption in analogy to the method described by Zilversmith et al. On day 13 of the experiment, the animals were sacrificed, and the animals' livers were removed for cholesterol analysis and preparation of microsomes. The 7α-hydroxylase activity was determined in the liver microsomes ex vivo by a modified method of Hylemon et al.

Combination of compound C1 with Caromax ®

| | | | | | Product |
|---|---|---|---|---|---|
| | | | | | mg/200 ml |
| 1 Teklad | | normal | n = 6 | −6 | — |
| | | ctr. I | | | |
| 2 Teklad | +0.1% CH | hyperlip. ctr. | n = 6 | −12 | — |
| | | (0.1% CH) | | | |
| 3 Teklad | +0.1% CH | 30 mg/kg/d C1 | n = 6 | −18 | 600 |
| 4 Teklad | +0.1% CH | 5% Caromax in the | n = 6 | −24 | |
| | | feed | | | |
| 5 Teklad | +0.1% CH | 30 mg/kg/d C1 + 5% | n = 6 | −30 | 600 |
| | | Caromax (feed) | | | |

The substances are dissolved in Solutol (50° C.) in a final concentration of 5%.
The solutions are then suspended with 0.4% potato starch.
Administration takes place 1 × a day with 10 ml/kg

Feed

Teklad 8604M CH: 030610M

Experimental Animals

Male Syrian hamsters (Mesocricetus auratus) supplied by Harlan 80-100 g at the start of adaptation

Measured Parameters

Feed consumption

Animal weight (weekly)

Safety parameters (CH; TG; ALAT/ASAT; AP; HDL/LDL)

Preliminary value and 2 days before end of the experiment (isoflurane anesthesia) by retroorbital blood sampling

Liver weight

Liver cholesterol (HPLC)=1×500 mg in EtOH/KOH (sample is also used for CH synthesis)

CYP7 activity (liver microsomes as group pool of 0.5 g each—preparation on day of experiment)

Cholesterol Synthesis

i.v. administration of ¹⁴C-octanoate 10 μCi/100 g of animal 1 hour before the end of the experiment (isoflurane anesthesia)

Removal of 2×500 mg of liver in EtOH/KOH

TABLE I

| | Cholesterol | | | Triglycerides | | | LDL-cholesterol | | | HDL-cholesterol | | |
|---|---|---|---|---|---|---|---|---|---|---|---|---|
| Feed/product | mmol/L | STD | % | mmol/L | STD | % | mmol/L | STD | % | mmol/L | STABW | % |
| Normal ctr. I | 2.91 | ±0.14 | 72 | 1.53 | ±0.24 | 105 | 0.46 | ±0.05 | 39 | 2.16 | ±0.08 | 86 |
| Hyperlip. ctr. (0.1% CH) | 4.02 | ±0.19 | 100 | 1.46 | ±0.34 | 100 | 1.17 | ±0.14 | 100 | 2.52 | ±0.15 | 100 |
| +0.1% CH | 3.58 | ±0.23 | 89 | 1.49 | ±0.16 | 102 | 0.88 | ±0.10 | 75 | 2.42 | ±0.23 | 96 |
| 30 mg/kg/d C1 | | | | | | | | | | | | |
| +0.1% CH | 3.63 | ±0.48 | 90 | 1.34 | ±0.58 | 92 | 1.05 | ±0.33 | 89 | 2.38 | ±0.34 | 95 |
| 5% Caromax in feed | | | | | | | | | | | | |
| +0.1% CH | 2.51 | ±0.33 | 62 | 1.34 | ±0.26 | 92 | 0.45 | ±0.08 | 39 | 1.82 | ±0.20 | 72 |
| 30 mg/kg/d C1 + 5% Caromax (Feed) | | | | | | | | | | | | |

| | Liver | | | | | |
|---|---|---|---|---|---|---|
| | Cholesterol | | | Sterol biosynthesis | | |
| Feed/product | mg/g | STD | % | dpm/g/h | STD | % |
| Normal ctr. I | 2.80 | ±0.37 | 10 | 409 | ±296 | 100 |
| Hyperlip. ctr. (0.1% CH) | 27.11 | ±6.04 | 100 | 50 | ±12 | 12 |
| +0.1% CH | 14.72 | ±2.16 | 54 | 73 | ±18 | 18 |
| 30 mg/kg/d C1 | | | | | | |
| +0.1% CH | 20.50 | ±3.73 | 76 | 45 | ±18 | 11 |
| 5% Caromax in feed | | | | | | |
| +0.1% CH | 4.14 | ±0.92 | 15 | 216 | ±114 | 53 |
| 30 mg/kg/d C1 + 5% Caromax (Feed) | | | | | | |

Abbreviations: 0.1% CH = 0.1% cholesterol in the feed
5% Caromax = 5% Caromax added to the feed; equivalent to a dose of 5000 mg/kg/day

Effect of ezetimibe (K00 04513) plus C1 on cholesterol absorption
Ezetimibe (K00 04513) is a cholesterol absorption inhibitor from Schering Plough

| | | | | | |
|---|---|---|---|---|---|
| 1 | Teklad | | Normal ctr. | n = 5 | −5 |
| 2 | Teklad | +0.1% CH | Cholesterol ctr. | n = 5 | −10 |
| 3 | Teklad | +0.1% CH | 0.1 mg/kg/d K 00 04513 | n = 5 | −15 |
| 4 | Teklad | +0.1% CH | 0.3 mg/kg/d K 00 04513 | n = 5 | −20 |
| 5 | Teklad | +0.1% CH | 1 mg/kg/d K 00 04513 | n = 5 | −25 |
| 6 | Teklad | +0.1% CH | 3 mg/kg/d C1 | n = 5 | −30 |
| 7 | Teklad | +0.1% CH | 10 mg/kg/d C1 | n = 5 | −35 |
| 8 | Teklad | +0.1% CH | 30 mg/kg/d C1 | n = 5 | −40 |
| 9 | Teklad | +0.1% CH | 0.1 mg/kg/d K 00 04513 + 10 mg/kg/d C1 | n = 5 | −45 |
| 10 | Teklad | +0.1% CH | 0.3 mg/kg/d K 00 04513 + 3 mg/kg/d C1 | n = 5 | −50 |
| 11 | Teklad | +0.1% CH | 0.1 mg/kg/d K 00 04513 + 3 mg/kg/d C1 | n = 5 | −55 |
| 12 | Teklad | +0.1% CH | 0.3 mg/kg/d K 00 04513 + 10 mg/kg/d C1 | n = 5 | −60 |

K00 04513 employed as stock solution (1 mg/ml in EtOH)
Substances are dissolved in 2% EtOH in a final concentration of 5%.
The solutions are then suspended with 0.4% potato starch.
Administration takes place 1× in the morning with 10 ml/kg
Feed: Teklad 8604M CH: 032201M
Experimental animals: Male Syrian hamsters (Mesocricetus auratus) supplied by Harlan
100-120 g at the start of adaptation
Measured parameters:
Feed consumption
Animal weight (weekly)
Liver weight
Safety parameters (CH; TG; ALAT/ASAT; AP; HDL/LDL)
Liver cholesterol (HPLC) = 1 × 500 mg in EtOH/KOH
CYP7 activity (liver microsomes as group pool of 0.5 g each - preparation on day of experiment)
Feces collected on day 5-7 for bile acid determination
Cholesterol absoroption
Oral administration of 2 μCi of ³H-sitosterol/1 μCi of ¹⁴C-cholesterol in 0.5 ml 1:1 tricaprin:tricaprylin
Feces collected on day 10-12
The feces are then dried and combusted in an Oximate (Packard) for isotope determination

TABLE II

| | | Plasma | | | | | | | | |
|---|---|---|---|---|---|---|---|---|---|---|
| | | parameter | | | | | | | | |
| | | Cholesterol | | | Triglycerides | | | LDL | | |
| Group | Feed/product | mmol/L | STD | % | mmol/L | STD | % | mmol/L | STD | % |
| 1 | Normal ctr. | 2.95 | ±0.18 | 72 | 1.76 | ±0.15 | 86 | 0.60 | ±0.09 | 54 |
| 2 | Cholesterol ctr. | 4.09 | ±0.18 | 100 | 2.04 | ±0.15 | 100 | 1.13 | ±0.20 | 100 |
| 3 | +0.1% CH | 3.73 | ±0.39 | 91 | 1.99 | ±0.18 | 98 | 1.06 | ±0.13 | 94 |
| | 0.1 mg/kg/d K 00 04513 | | | | | | | | | |
| 4 | +0.1% CH | 2.99 | ±0.40 | 73 | 1.87 | ±0.41 | 92 | 0.40 | ±0.07 | 35 |
| | 0.3 mg/kg/d K 00 04513 | | | | | | | | | |
| 5 | +0.1% CH | 2.53 | ±0.29 | 62 | 1.79 | ±0.23 | 88 | 0.23 | ±0.02 | 20 |
| | 1 mg/kg/d K 00 04513 | | | | | | | | | |
| 6 | +0.1% CH | 3.92 | ±0.46 | 96 | 1.84 | ±0.31 | 90 | 0.98 | ±0.24 | 87 |
| | 3 mg/kg/d C1 | | | | | | | | | |
| 7 | +0.1% CH | 3.70 | ±0.22 | 90 | 2.35 | ±0.40 | 116 | 0.78 | 0.20 | 69 |
| | 10 mg/kg/d C1 | | | | | | | | | |
| 8 | +0.1% CH | 3.66 | ±0.31 | 89 | 2.02 | ±0.47 | 99 | 0.80 | ±0.04 | 71 |
| | 30 mg/kg/d C1 | | | | | | | | | |
| 9 | +0.1% CH | 2.81 | ±0.10 | 69 | 1.51 | ±0.33 | 74 | 0.55 | ±0.10 | 49 |
| | 0.1 mg/kg/d K 00 04513 + | | | | | | | | | |
| | 10 mg/kg/d C1 | | | | | | | | | |
| 10 | +0.1% CH | 2.73 | ±0.39 | 67 | 1.71 | ±0.44 | 84 | 0.31 | ±0.10 | 28 |
| | 0.3 mg/kg/d K 00 04513 + | | | | | | | | | |
| | 3 mg/kg/d C1 | | | | | | | | | |
| 11 | +0.1% CH | 2.96 | ±0.19 | 72 | 1.82 | ±0.25 | 89 | 0.62 | ±0.15 | 55 |
| | 0.1 mg/kg/d K 00 04513 + | | | | | | | | | |
| | 3 mg/kg/d C1 | | | | | | | | | |
| 12 | +0.1% CH | 2.29 | ±0.35 | 56 | 0.99 | ±0.40 | 49 | 0.19 | ±0.06 | 17 |
| | 0.3 mg/kg/d K 00 04513 + | | | | | | | | | |
| | 10 mg/kg/d C1 | | | | | | | | | |

| | | | | | | | | CH | |
|---|---|---|---|---|---|---|---|---|---|
| | | | | | Liver | | | absorption | |
| | | HDL | | | Cholesterol | | | % | |
| Group | Feed/product | mmol/L | STD | % | mg/g | STD | % | absorption | % of ctr. |
| 1 | Normal ctr. | 1.78 | ±0.18 | 83 | 3.73 | ±0.67 | 34 | 49.0 | ±100.0 |
| 2 | Cholesterol ctr. | 2.15 | ±0.13 | 100 | 11.02 | ±0.55 | 100 | 50.4 | ±102.9 |
| 3 | +0.1% CH | 1.98 | ±0.23 | 92 | 11.52 | ±1.27 | 105 | 47.4 | ±96.8 |
| | 0.1 mg/kg/d K 00 04513 | | | | | | | | |
| 4 | +0.1% CH | 1.92 | ±0.21 | 89 | 2.00 | ±0.12 | 18 | 15.6 | ±31.8 |
| | 0.3 mg/kg/d K 00 04513 | | | | | | | | |
| 5 | +0.1% CH | 1.71 | ±0.19 | 80 | 1.78 | ±0.08 | 16 | 5.8 | ±11.9 |
| | 1 mg/kg/d K 00 04513 | | | | | | | | |
| 6 | +0.1% CH | 2.20 | ±0.14 | 102 | 10.99 | ±1.82 | 100 | 39.6 | ±80.9 |
| | 3 mg/kg/d C1 | | | | | | | | |
| 7 | +0.1% CH | 2.00 | ±0.17 | 93 | 9.50 | ±1.19 | 86 | 49.1 | ±100.1 |
| | 10 mg/kg/d C1 | | | | | | | | |
| 8 | +0.1% CH | 2.02 | ±0.28 | 94 | 7.18 | ±0.60 | 65 | 38.3 | ±78.2 |
| | 30 mg/kg/d C1 | | | | | | | | |
| 9 | +0.1% CH | 1.74 | ±0.10 | 81 | 2.71 | ±0.43 | 25 | 17.1 | ±34.9 |
| | 0.1 mg/kg/d K 00 04513 + | | | | | | | | |
| | 10 mg/kg/d C1 | | | | | | | | |
| 10 | +0.1% CH | 1.84 | ±0.16 | 86 | 2.15 | ±0.48 | 20 | 10.4 | ±21.2 |
| | 0.3 mg/kg/d K 00 04513 + | | | | | | | | |
| | 3 mg/kg/d C1 | | | | | | | | |
| 11 | +0.1% CH | 1.75 | ±0.12 | 82 | 2.82 | ±1.02 | 26 | 23.2 | ±47.4 |
| | 0.1 mg/kg/d K 00 04513 + | | | | | | | | |
| | 3 mg/kg/d C1 | | | | | | | | |
| 12 | +0.1% CH | 1.71 | ±0.21 | 80 | 1.92 | ±0.31 | 17 | 9.5 | ±19.5 |
| | 0.3 mg/kg/d K 00 04513 + | | | | | | | | |
| | 10 mg/kg/d C1 | | | | | | | | |
| K 00 04513 = ezetimibe cholesterol absorption inhibitor, Schering Plough It is evident from the tables that the derivative of formula In combination with Caromax ® and ezetimibe show a synergistic effect on the plasma parameters. Thus, for example, treatment with 0.1 mg/kg K 00 04513 (line 3) reduces the LDL-cholesterol to 94%, and treatment with 3 mg/kg C1 (line 6) reduces | | | | | | | | | |
| # the LDL-cholesterol to 87%. Combination treatment with 0.1 mg/kg K 00 045 13 and 3 mg/kg C1 (line 10) reduces the LDL-cholesterol to 28%. | | | | | | | | | |

The present invention may be embodied in other specific forms without departing from the spirit or essential attributes thereof.

We claim:

1. A composition of matter comprising a derivative of formula I 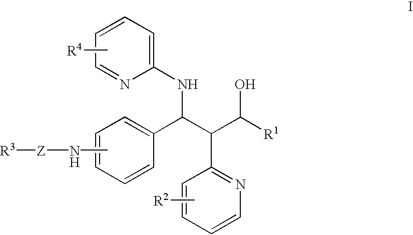
in which
R¹ is phenyl optionally substituted by one to three mutually independent radicals, or heteroaryl optionally substituted by one to three mutually independent radicals, where the radicals are selected from the group consisting of fluorine, chlorine, bromine, iodine, OH, CF₃, —NO₂, —CN, (C₁-C₈)-alkoxy, (C₁-C₈)-alkyl, —NH₂, —NH—R⁹, —N(R⁹)R¹⁰, —CHO, —COOH, —COOR¹¹, —(C═O)—R¹², (C₁-C₆)-alkyl-OH, (C₁-C₆)-alkyl(—OH)-phenyl, (C₁-C₆)-alkyl-CF₃, (C₁-C₆)-alkyl-NO₂, (C₁-C₆)-alkyl-CN, (C₁-C₆)-alkyl-NH₂, (C₁-C₆)-alkyl-NH—R⁹, (C₁-C₆)-alkyl-N(R⁹)R¹⁰, (C₁-C₆)-alkyl-CHO, (C₁-C₆)-alkyl-COOH, (C₁-C₆)-alkyl-COOR¹¹, (C₁-C₆)-alkyl-(C═O)—R¹², —O—(C₁-C₆)-alkyl-OH, —O—(C₁-C₆)-alkyl-CF₃, —O—(C₁-C₆)-alkyl-NO₂, —O—(C₁-C₆)-alkyl-CN, —O—(C₁-C₆)-alkyl-NH₂, —O—(C₁-C₆)-alkyl-NH—R⁹, —O—(C₁-C₆)-alkyl-N(R⁹)R¹⁰, —O—(C₁-C₆)-alkyl-CHO, —O—(C₁-C₆)-alkyl-COOH, —O—(C₁-C₆)-alkyl-COOR¹¹, —O—(C₁-C₆)-alkyl-(C═O)—R¹², —N—SO₃H, —SO₂—CH₃, —O—(C₁-C₆)-alkyl-O—(C₁-C₆)-alkyl-phenyl, (C₁-C₆)-alkylthio, or pyridyl, and wherein one or more hydrogen atom(s) in the alkyl radical is optionally replaced by fluorine, and wherein for the phenyl or pyridyl radical is optionally monosubstituted by methyl, methoxy or halogen;
R² is H, —OH, —CH₂OH, —OMe, —CHO, or —NH₂;
R³ is saccharide residue, disaccharide residue, trisaccharide residue, or tetrasaccharide residue,
wherein the saccharide residue, disaccharide residue, trisaccharide residue or tetrasaccharide residue is optionally substituted by one or more saccharide protective groups selected from the group of HO—SO₂— and(HO)₂—PO—;
R⁴ is H, methyl, F, —OMe;
R⁹ to R¹² are, independently of one another, H or C₁-C₈-alkyl;
Z is —NH—C₀-C₁₆-alkyl-C═O—, —O—C₀-C₁₆-alkyl-C═O—, —(C═O)ₘ-C₁-C₁₆-alkyl-(C═O)ₙ—, amino acid residue, or diamino acid residue, wherein the amino acid residue or diamino acid residue is optionally substituted by one or more amino acid protective groups, or a covalent bond;
n is 0 or 1;or
m is 0 or 1;and
a pharmaceutically acceptable salt or physiologically functional derivatives thereof, with an other active ingredient, or a pharmaceutically acceptable salt or physiologically functional derivatives thereof. The invention is also directed to the use of the combination product, pharmaceutical composition comprising the combination product and method for preparing the pharmaceutical composition.

2. The composition of matter according to claim 1, wherein
R¹ is phenyl, thiazolyl, oxazolyl, and isoxazolyl, and wherein for the phenyl thiazolyl, oxazolyl, and isoxazolyl are optionally substituted by one to two fluorine, chlorine, bromine, or (C₁-C₈)-alkyl;
R³ is a saccharide residue selected from the group consisting of 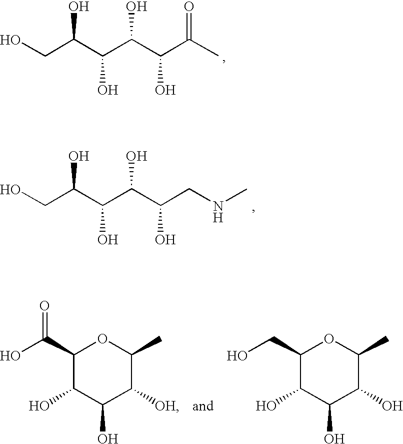
wherein the saccharide residue is optionally substituted by one or more HO—SO₂—; and
Z is —NH—C₆-C₁₂-alkyl-C═O—, —O—C₆-C₂-alkyl-C═O—, —(C═O)ₘ-C₆-C₁₂-alkyl-(C═O)ₙ—; or
the pharmaceutically acceptable acid addition salt thereof.

3. The composition of matter according to claim 1, wherein the derivative of formula I is 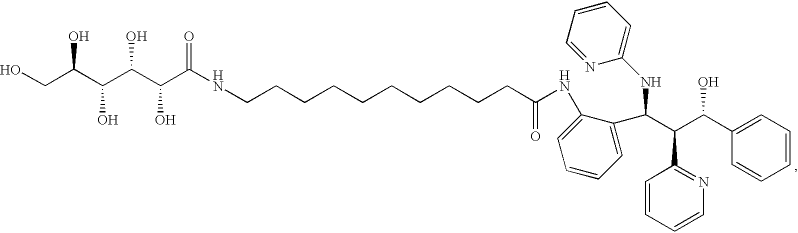
the pharmaceutically acceptable acid addition salt thereof.

4. The composition of matter according to one of claims 1 to 3, wherein the other active ingredient is an antidiabetic, hypoglycemic active ingredient, HMG-CoA reductase inhibitor, cholesterol absorption inhibitor, PPAR gamma agonist, PPAR alpha agonist, PPAR alpha/gamma agonist, fibrate, MTP inhibitor, bile acid absorption inhibitor, CETP inhibitor, polymeric bile acid adsorbent, LDL receptor inducer, ACAT inhibitor, antioxidant, lipoprotein lipase inhibitor, ATP-citrate lyase inhibitor, squalene synthetase inhibitor, lipoprotein(a) antagonist, lipase inhibitor, insulin, sulfonylurea, biguanide, meglitinide, thiazolidinedione, α-glucosidase inhibitor, active ingredient acting on the ATP-dependent potassium channel of the beta cells, CART agonist, NPY agonist, MC4 agonist, orexin agonist, H3 agonist, TNF agonist, CRF agonist, CRF BP antagonist, urocortin agonist, β3 agonist, MSH (melanocyte-stimulating hormone) agonist, CCK agonist, serotonin reuptake inhibitor, mixed serotoninergic and noradrenergic compound, 5HT agonist, bombesin agonist, galanin antagonist, growth hormone, growth hormone-releasing compound, TRH agonist, uncoupling protein 2 or 3 modulator, leptin agonist, DA agonist, lipase/amylase inhibitor, PPAR modulator, RXR modulator, TR-β agonist or amphetamine.

5. The composition of matter according to one of claims 1 to 3, wherein the other active ingredient is a compound that normalizes lipid metabolism.

6. The composition of matter according to one of claims 1 to 3, wherein the other active ingredient is a compound that normalizes lipid metabolism selected from the group consisting of statins, glitazones, PPAR alpha agonists, cholestyramine, colestipol, colesevelam, adsorbent resins, fibrates, gemfibrozil, cholesterol absorption inhibitors, ezetimibe, tiqueside, pamaqueside, CETP inhibitors, MTP inhibitors, LDL receptor inducers, lipase inhibitors, and orlistat.

7. The composition of matter according to one of claims 1 to 3, wherein the other active ingredient is a cholesterol absorption inhibitor.

8. The composition of matter according to claim 7, wherein the cholesterol absorption inhibitor is ezetimibe, tiqueside or pamaqueside.

9. The composition of matter according to one of claims 1 to 3, comprising Caromax® as other active ingredient.

10. A method for effecting the prophylaxis or treatment of a lipid metabolism disorder or metabolic syndrome in a patient comprising administering a pharmaceutically effective amount of the composition of matter according to one of claims 1 to 3 to the patient.

11. The method of claim 10 wherein the pharmaceutically effective amount of the composition of matter is provided for by the combination of a pharmaceutically effective amount or a subclinical pharmaceutically effective amount of the compound of formula I and a pharmaceutically effective amount or a subclinical pharmaceutically effective amount of the other active ingredient of the composition of matter, such that the combination results in the amount of the composition of matter being pharmaceutically effective.

12. A method for effecting the prophylaxis or treatment of hyperlipidemia in a patient comprising administering a pharmaceutically effective amount of the composition of matter according to one of claims 1 to 3 to the patient..

13. The method of claim 12 wherein the pharmaceutically effective amount of the composition of matter is provided for by the combination of a pharmaceutically effective amount or a subclinical pharmaceutically effective amount of the compound of formula I and a pharmaceutically effective amount or a subclinical pharmaceutically effective amount of the other active ingredient of the composition of matter, such that the combination results in the amount of the composition of matter being pharmaceutically effective.

14. A method for effecting the prophylaxis or treatment of arteriosclerotic manifestations in a patient comprising administering a pharmaceutically effective amount of the composition of matter according to one of claims 1 to 3 to the patient.

15. The method of claim 14 wherein the pharmaceutically effective amount of the composition of matter is provided for by the combination of a pharmaceutically effective amount or a subclinical pharmaceutically effective amount of the compound of formula I and a pharmaceutically effective amount or a subclinical pharmaceutically effective amount of the other active ingredient of the composition of matter, such that the combination results in the amount of the composition of matter being pharmaceutically effective.

16. A method for effecting the prophylaxis or treatment of a physiological condition as described herein in a patient comprising administering a pharmaceutically effective amount of the composition of matter according to one of claims 1 to 3 to the patient whereby the administering is effected by administering the compound of formula I and the other active ingredient of the composition of matter closely in time.

17. The method of claim 16 wherein closely in time means within 10 minutes.

18. A method for effecting the prophylaxis or treatment of a lipid metabolism disorder in a patient comprising administering a pharmaceutically effective amount of the composition of matter according to one of claims 1 to 3 to the patient whereby the administering is effected by administering the compound of formula I and the other active ingredient of the composition of matter closely in time.

19. The method of claim 18 wherein closely in time means within 10 minutes.

20. A pharmaceutical composition comprising, a pharmaceutically acceptable carrier, and a pharmaceutically effective amount or a subclinical pharmaceutically effective amount of the compound of formula I according to one of claims 1 to 3 and a pharmaceutically effective amount or a subclinical pharmaceutically effective amount of the other active ingredient of the composition of matter, such that the combination results in the amount of the composition of matter being pharmaceutically effective.

21. A process for producing a pharmaceutical composition of the composition of matter as claimed in one of claims 1 to 3, comprising mixing the compound of formula I and the other active ingredient of the composition of matter with a pharmaceutically suitable carrier and converting this mixture into a form suitable for administration.
